(19) United States
(12) Patent Application Publication
(10) Pub. No.: US 2008/0294232 A1
(43) Pub. Date: Nov. 27, 2008

(54) MAGNETIC CELL DELIVERY

(76) Inventor: Raju R. Viswanathan, St. Louis, MO (US)

Correspondence Address: Bryan K. Wheelock, Suite 400, 7700 Bonhomme, St. Louis, MO, 63105, US

(21) Appl. No.: 12/121,774

(22) Filed: May 15, 2008

Related U.S. Application Data
(60) Provisional application No. 60/939,614, filed on May 22, 2007.

(51) Int. Cl. A61F 2/06; A61N 2/00

(52) U.S. Cl. 623/1.11; 600/12

(57) ABSTRACT

Systems and methods of delivering magnetically loaded cells to target areas within a patient are described. Cells rendered magnetically attractable by being loaded with magnetic microparticles are delivered from a hollow interventional device distal tip and attracted towards a previously placed implant. Implants, such as stents, are magnetized by application of a magnetic field sequence; magnetized cells are attracted by the local magnetic domains and associated field gradients within the implant, and adhere to and are retained by the local tissues, such as tissue protrusions through a stent struts. Application of a magnetic field or field gradient sequence concurrently with the magnetic cell delivery facilitates pulling the cells away from the lumen axis and towards the implant surface and vessel or organ walls.

### CROSS-REFERENCE TO RELATED APPLICATIONS

This application claims priority to U.S. Provisional Patent Application Ser. No. 60/939,614, filed May 22, 2007, the entire disclosure of which is incorporated herein.

### FIELD OF THE INVENTION

This invention relates to methods and systems for magnetically facilitating delivery of cells to target structures, implants, or organs. In particular, a method of guiding magnetized cells to a target by the application of a magnetic field or a magnetic field gradient is disclosed.

### BACKGROUND OF THE INVENTION

Minimally invasive intervention systems include navigation systems, such as the Niobe™ magnetic navigation system developed by Stereotaxis, St. Louis, Mo. Such systems typically comprise an imaging means for real-time guidance and monitoring of the intervention; additional feedback can be provided by a three-dimensional (3D) localization system that allows real time determination of the catheter or interventional device tip position and orientation with respect to the operating room and, through co-registered imaging, with respect to the patient.

The availability of methods and systems for safe, efficient minimally invasive interventions have greatly impacted and changed the practice of cardiac treatment delivery in the last decade. The treatment of a number of cardiac disorders has become possible without requiring open heart surgery. In particular, progress in vascular interventions such as crossing and opening of occluded and stenosed arteries, placement of stents, and local delivery of therapeutic agents have significantly helped in reducing the morbidity and mortality related to coronary arteries impairment and associated cardiac ischemia.

As methods and technologies evolve, treatment is considered for smaller and narrower arteries in an attempt at both prolonging life and improving quality of life. Challenges associated with treatment of arteries with a diameter in the range 2 to 5-mm, such as the coronaries, include the rejection of graft; the re-occlusion of vessels, including stented vessels; and the resulting frequent need to re-intervene at sites previously treated.

Recently, studies conducted by researchers at the Mayo clinic and elsewhere have demonstrated the feasibility of magnetically localizing cells at the site of a stented vessel wall in a large animal model (Pislaru SV et al., Magnetically Targeted Endothelial Cell Localization in Stented Vessels, Journal of the American College of Cardiology, Vol. 48, No. 9, 2006), incorporated herein by reference. In particular, cell localization was demonstrated using paramagnetic nickel (Ni) coating on stents magnetized prior placement by a 0.5 T magnetic field. U.S. patent application Ser. No. 11/210,173, entitled “Magnetically-controllable delivery system for therapeutic agents”, incorporated herein by reference, describes methods of magnetically delivering particles to a target area within a subject body. U.S. patent application Ser. No. 10/081,770 entitled “Methods and apparatuses for delivering a medical agent to a medical implant,” published as U.S. publication 2002/0133225 on Sep. 19, 2002 and now abandoned, incorporated herein by reference, discloses the use of a ferromagnetic implant capable of magnetization.

### SUMMARY OF THE INVENTION

Embodiments of the present invention provide devices and systems for the magnetic delivery of cells to specific targets, and methods of using such devices and systems.

More specifically, embodiments of this invention relate to methods of delivering magnetized cells to specific targets and methods of retaining the cells at a selected target. Such methods include the use of magnetizing magnetic fields, motion control magnetic-field gradients, and associated medical devices. The methods can further include the application of magnetic field or field gradient sequences during the cell delivery at target site(s), and associated medical devices comprising specific designs and device composition for improved cell capture.

Further areas of applicability of the embodiments of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1-A is a schematic diagram showing a patient positioned in a projection imaging and interventional system for a minimally invasive procedure such as a coronary arteries diagnostic and therapeutic intervention;

FIG. 1-B schematically illustrates an interventional device distal end being navigated through one of the patient's vessels in the vicinity of an implant such as an arterial stent;

FIG. 1-C schematically presents an interventional distal end located upstream from a stent in an artery, delivering magnetized cells to the stent through the blood flow;

FIG. 2-A presents a functional block diagram of a preferred embodiment of the present invention as applied to the delivery of cells to a target organ wall;

FIG. 2-B shows a functional block diagram pertaining to the design and manufacture of medical implants to be used by some preferred methods in accordance with the present invention;

FIG. 3 presents in more detail an example of a delivery catheter, magnetized stent, and delivery of magnetized cells according to one embodiment of the present invention using sequences of magnetization fields;

FIG. 4 describes a magnetizing field sequence B(t) applied to a stent or implant;

FIG. 5 presents a magnetizing field gradient sequence grad B′(t) applied to a stent or implant; and

FIG. 6 presents a flow chart of a preferred embodiment of the magnetic cell delivery method as applied to the interventional system of FIG. 1.

Corresponding reference numerals indicate corresponding parts throughout the several views of the drawings.

### DETAILED DESCRIPTION OF THE INVENTION

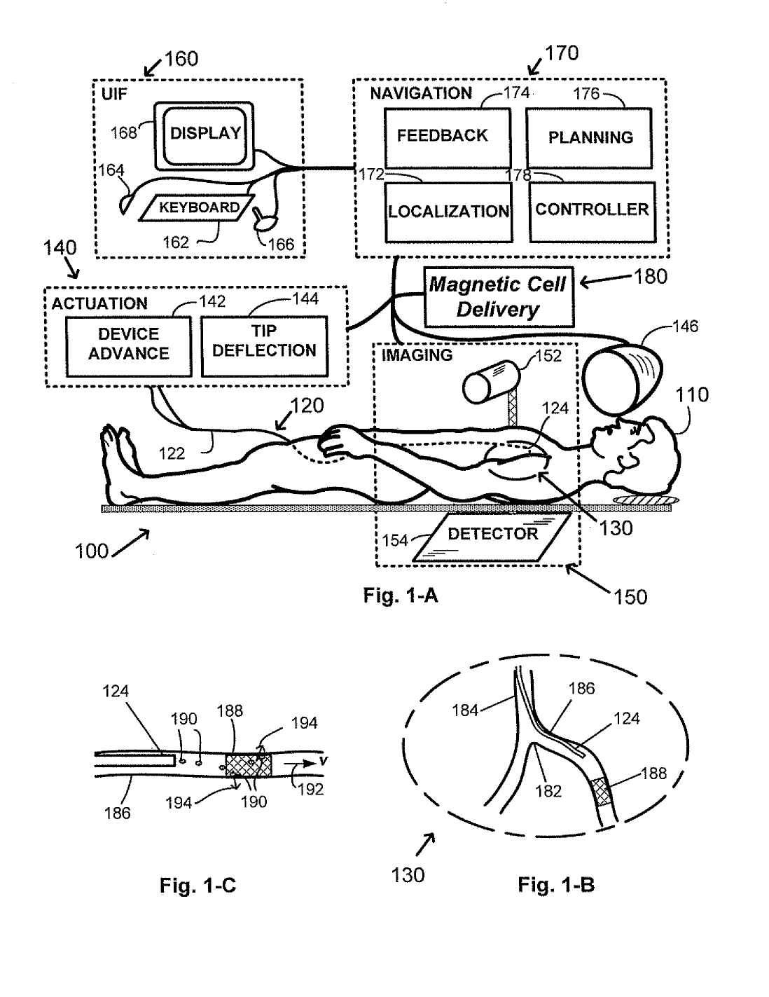

As illustrated in FIG. 1-A, a patient 110 is positioned within a remotely actuated, computer controlled interventional system 100. An elongated navigable medical device 120 having a proximal end 122 and a distal end 124 is provided for use in the interventional system 100 and the medical device is inserted into a blood vessel of the patient and navigated to an intervention volume 130.

A means of applying force or torque to advance or orient the device distal end 124 is provided, as illustrated by actuation block 140 comprising a component 142 capable of precise proximal device advance and retraction and a tip deflection component 144. The actuation sub-system for tip deflection may be one of (i) a mechanical pull-wire system; (ii) a hydraulic or pneumatic system; (iii) an electrostrictive system; (iv) a magnetostrictive system; (v) a magnetic system; or (vi) other navigation system as known in the art. For illustration of a preferred embodiment, in magnetic navigation a magnetic field externally generated by magnet(s) assembly 146 orients a small magnetically responsive element (not shown) located at or near the device distal end 124.

Real time information is provided to the physician by an imaging sub-system 150, for example an x-ray imaging chain comprising an x-ray tube 152 and a digital x-ray detector 154, to facilitate planning and guidance of the procedure. Additional real-time information, such as distal tip position and orientation may be supplied by use of a three-dimensional (3D) device localization sub-system, such as comprising a set of electromagnetic wave receivers located at the device distal end (not shown), and associated external electromagnetic wave emitters (not shown); or other localization device with similar effect such as an electric field-based localization system that measures local fields induced by an externally applied voltage gradient. In the latter case the conducting body of a wire within the device itself carries the signal recorded by the tip electrode to a proximally located localization system.

The physician provides inputs to the navigation system through a user interface (UIF) sub-system 160 comprising user interfaces devices such as keyboard 162, mouse 164, joystick 166, display 168, and similar input or output devices. Display 168 also shows real-time image information acquired by the imaging system 150 and localization information acquired by the three-dimensional localization system. UIF sub-system 160 relays inputs from the user to a navigation sub-system 170 comprising 3D localization block 172, feedback block 174, planning block 176, and controller 178.

Navigation control sequences are determined by the planning block 176 based on inputs from the user, and also possibly determined from pre-operative or intra-operative image data and localization data from a localization device and sub-system, as described above and processed by localization block 172, and alternatively or additionally, real-time imaging or additional feedback data processed by feedback block 174. The navigation control sequence instructions are then sent to controller 178 that actuates interventional device 120 through actuation block 140 to effect device advance or retraction and tip deflection.

Other navigation sensors might include an ultrasound device or other device appropriate for the determination of distances from the device tip to surrounding tissues, or for tissue characterization. Further device tip feedback data may include relative tip and tissues positions information provided by a local intra-operative imaging system, and predictive device modeling and representation. Such device feedback in particular, enables remote control of the intervention. In closed-loop implementations, the navigation sub-system 170 automatically provides input commands to the device advance/retraction 142 and tip orientation 144 actuation components based on feedback data and previously provided input instructions. In semi closed-loop implementations, the physician fine-tunes the navigation control, based in part upon displayed information and possibly other feedback data, such as haptic force feedback. Control commands and feedback data may be communicated from the user interface 160 and navigation sub-system 170 to the device and from the device back to navigation sub-system 170 and the user through cables or other means, such as wireless communications and interfaces. Additionally, FIG. 1-A schematically shows magnetic cell delivery block 180 that performs specific functions in various embodiments of the present invention. Cell delivery block 180 applies to magnetic navigation system, such as that illustrated in FIG. 1-A, and more generally to any medical navigation device that also comprises an external magnet for the generation of specific magnetic field sequences during cell delivery, as described in this disclosure.

FIG. 1-B schematically shows the distal end 124 of interventional device 120 having progressed through a branch 182 of the coronary arterial tree 184 into the left branch 186. There the device distal end is navigated up-flow toward the vicinity of an implant such as a stent 188.

In the context of this invention, FIG. 1-C shows the distal end 124 of interventional device 120 located upstream from implant 188 in arterial branch 186. Magnetized cells 190 (not to scale) are released through device 120 and float downstream toward the stent or implant 188 at average velocity ν 192 as determined by local hemodynamics (also function of the cardiac cycle phase). Upon passing through stent or implant 188, magnetized cells 190 are attracted towards the local, randomly oriented, magnetic fields and field gradients 194 (not to scale) that are present within and in the immediate vicinity of the stent or implant after magnetization. These fields ensure that a significant fraction of the delivered magnetized cells are attracted to and attach onto the tissue structures locally protruding through the stent or implant struts. The mechanism of magnetic attraction enable local cell retention for a time period sufficient for natural tissue mechanisms to “bond” the cells to the local tissue, thereby leading to the generation of an endothelial cell layer that has the natural characteristic of the arterial wall.

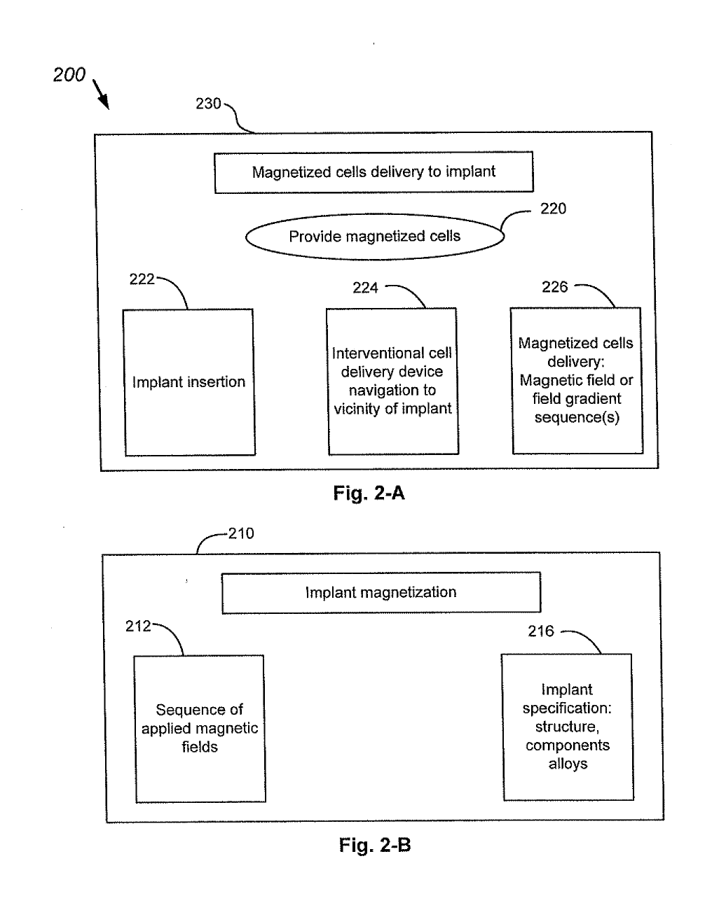

FIG. 2-A, 230, illustrates a block diagram for the functionality of block 180. Magnetized cells of specific tissue characteristics and magnetic properties are selected for a given application, 220. Cells can be magnetically loaded, for example by labeling with micro-spheres; other magnetization means include the use of hollow magnetic volumes that contain specific cells, for example therapeutic cells; and of other similar magnetic delivery vehicles; all such vehicles thereafter also denoted by the term “magnetic particles.” Following insertion of an implant at the target site, block 222, an interventional cell delivery device is navigated toward the vicinity of and upstream from the implant, block 224. The interventional device navigation to the interventional site maybe effected by a magnetic navigation system, such as described in FIG. 1-A, or by any other navigation system as known in the art. Magnetized cells are injected at the interventional device proximal end and delivered upstream from the implant in step 226.

In one preferred embodiment of the present invention, during magnetized particles injection, a sequence of magnetic fields is applied to the implant volume; such sequence leads to the generation of local magnetization domains with local magnetization preferably oriented along the instantaneous direction of the field. The externally generated field is sufficient to induce magnetization of the magnetic domains in the implant. In such an embodiment, the time sequence of applied fields, preferably oriented generally in a plane perpendicular to the implant or stent local long axis, lead to a relatively uniform deposition of magnetized particles onto the implant and onto the local tissues protruding through the implant structures. Further, the time sequencing of fields can yield a uniform cell deposition pattern regardless of domain size of the magnetic domains; without sequencing, larger domain sizes can lead to an effective bulk magnetization of the entire implant, leading to non-uniform cell deposition.

In another preferred embodiment of the present invention, during magnetized particles injection, a sequence of magnetic field gradients is applied to the implant volume; in such an application, the magnetized particles are pulled by the gradients with an intensity proportional to both the particles magnetic moment and the local field gradient. Ferromagnetic particles generally present a magnetic moment independent from the applied field, while for paramagnetic particles the moment is itself proportional to the applied field magnitude. Generally free particles will tend to orient such that their magnetic moment is parallel to the field, and the pulling force will apply in the direction where the magnetic field magnitude increases. Preferably, the gradients are applied in a plane generally perpendicular to the local implant long axis, in such a way that the magnetized particles are attracted toward the implant surface and therefore, toward the local tissues protruding through the implant structures. As the direction of the magnetic gradients is changed as a function of time within such a plane, a relatively angularly uniform distribution of magnetized particles is achieved on the vessel or organ wall onto which the implant surface lies.

FIG. 2-B describes steps in the manufacture of implants per specific design requirements. Depending on the clinical application, the size of the implant, the target anatomy and surrounding tissues, the volume of material available for magnetization, and the size of the magnetic carriers, such as micro-spheres that can be safely and sustainedly loaded onto specific target cells, specifications for the implant are derived, block 216. Other parameters may also be considered in designing the implant, such as the maximum expected blood velocity and associated shear forces; the tortuosity of the vessel; the likelihood of plaque presence, inflammation, or other clinically relevant circumstances. The method comprises magnetizing an implant, block 210, for example, through application of an appropriate sequence of magnetic fields, 212.

In one embodiment, during the magnetization process, it is desirable to create a high density of small local magnetic domains (paramagnetic or ferromagnetic) to create a sufficient number of magnetic dipoles on the stent or implant surface; such a distribution helping to ensure that the magnetic cell deposition process described above achieves a high degree of uniformity on the implant. If the domain size is large, the flux distributions through the domains lead to the generation of a macro-magnetic field and bulk magnetic poles, such that cell accumulation tends to occur preferably at both south and north magnetic poles.

Processes available as known in the art to induce domain creation and distribution on a suitable scale include electroplating, etching, or sputtering to magnetically coat the medical implant or device with a material such as nickel (Ni), platinum-cobalt (PtCo), platinum-iron (PtFe), or iron oxides. Should the magnetic coating not be biocompatible, various encapsulating methods as known in the art, such as dip coating or vapor deposition, can be used to deposit a protective polymer layer. Various design parameters 216 including the device shape, structure, density, choice of materials, layering, and manufacturing processes, are considered in the specification of implants or medical devices that are capable of being appropriately magnetized according to the methods of the present invention.

The favorable magnetization properties of PtCo enable optimization of magnetic coating to be responsive to a magnetic field of magnitude in the range 0.05 T to 5 T: the magnetization B obtained in applied fields H is non-linear, and B reaches saturation at a relatively small applied field magnitude, preferably in the range of 0.05 to 0.5 T. In one embodiment, even when the applied field magnitude returns to zero, the remaining magnetization Bᵣ is high. In this embodiment, the coercive field H_{c} necessary to return the magnetization to zero is high, indicating that after initial magnetization the material is likely to retain magnetization even in the presence of applied opposing fields as described previously. More preferably, the properties of the PtCo alloy, pattern of deposition, and volume of material enable the magnetic coating to be responsively and durably magnetized in a magnetic field in the range of 0.05 T to 0.5 T. For instance, masked electro-deposition can be used to create a suitably arrayed distribution of suitably small magnetic domains.

Studies have indicated that micro-spheres in the range 0.3 to 0.9-μm are well taken-up and tolerated by cells, in particular, by endothelial cells. Alternatively, “needles” or “ellipsoids” with aspect ratio d×l with d in the range 0.05 to 0.5 micron and l about 0.3 to 0.9 microns can be used as well; such “needles” can be obtained for example, by spray drying magnetic material under gravity, or under the presence of an applied electric or magnetic field. Shaped particles can be ferromagnetic or paramagnetic, as is known in the art.

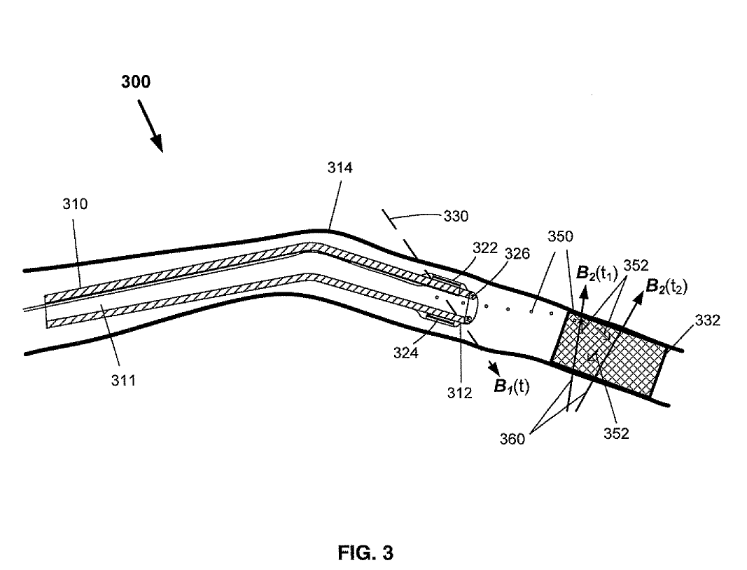

To further illustrate the invention, FIG. 3 presents a catheter or interventional device 310 having a lumen 311, a proximal end (not shown), and a distal end 312. In the figure, distal end 312 has progressed past a vessel bend 314 through a combination of proximal end advance and distal end magnetic steering. In this particular embodiment, distal end 312 comprises a set of electromagnets, three of which are shown as 322, 324, and 326. During navigation, the electromagnets are activated as necessary to generate a local tip magnet B′ (not shown) that interacts with an externally generated applied magnetic field B₁(t), 330. The fields B₁(t) and B′(t) are chosen as a function of the anatomy and local catheter tip orientation to facilitate navigation, for example to facilitate device progress past bend 314. When the distal end of the catheter is in place near to and upstream from an implant 332 magnetically loaded cells 350 are proximally injected and exit the device distal tip in the proximity of implant 332. Upon passing through the implant lumen, the magnetic particles are attracted by the local magnetic gradients 352 that have been induced in the material by prior magnetization; in various embodiments, implant magnetization occurs prior to implant delivery, prior to cell delivery, during cell delivery, or a combination thereof. In a preferred embodiment, applying a magnetic field sequence B₂(t) 360 during delivery increases the local domain magnetization and therefore the associated local gradients; the logistics of the intervention, patient positioning, cell delivery, and magnetic field application are such that simultaneous or near simultaneous field application is favorable. Further, it is also possible to dynamically apply magnetic field gradients during the intervention, the gradients (not shown) being such that the magnetized particles are pulled away from the vessel lumen and toward the walls, where they become attached to either the implant or to local tissues protruding through the implant.

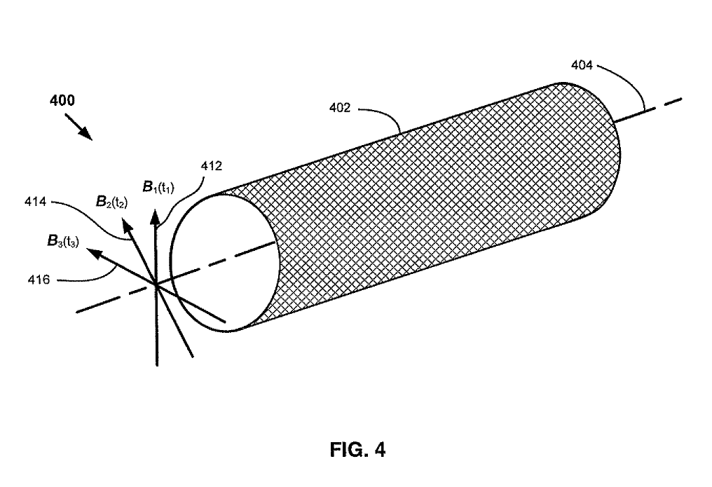

In an alternate preferred embodiment, and as illustrated generally in FIG. 4 by numeral 400, it is possible to work with implants coated with relatively large magnetic domains. Upon magnetization with a single applied magnetic field, a surface dipole distribution that is mostly bipolar is generated. By using a suitably changing sequence of applied magnetic fields soon after or preferably during magnetized cells injection, a sequence of bipolar surface gradients and associated bipolar cell distributions will result in a relatively uniform distribution of magnetized cells. Accordingly FIG. 4 describes the use of such an applied field sequence: knowing the orientation of implant 40a within the patient, as for example, available from interventional imaging and/or from having delivered the implant in a previous phase of the same intervention, the external magnet(s) are oriented, such that the applied fields are essentially orthogonal to the implant main axis 404. A sequence of transverse fields application, three fields being shown by 412, 414, and 416 in the figure, ensures more uniformly distributed domain magnetic fields, and accordingly more uniformly distributed magnetic cells following cell injection and distribution. Even if the magnetic coating contains large magnetic domains, the application of a magnetization field at a series of angles around axis 404 will help to ensure a uniform distribution of magnetic cells. The stent 402 itself could be made of a bulk paramagnetic/ferromagnetic material. In an embodiment of the present invention using Stereotaxis' Niobe™ system, the magnets are brought in from a semi-stowed position. It is noted that in this application, the applied field(s) need not be strictly in a plane orthogonal to the local stent or device long axis. However it is important that a significant orthogonal field component be present to help ensure a more uniform magnetic particle deposition on the stent, and therefore it is desirable to avoid applying fields that are essentially collinear or make a shallow angle with the implant main axis 404.

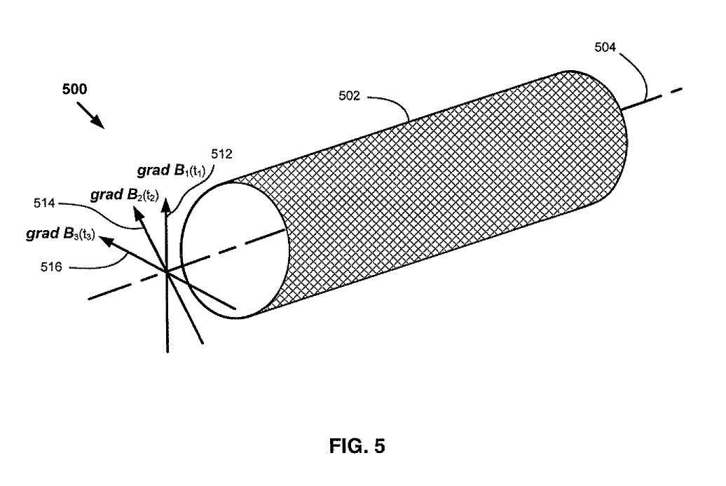

FIG. 5 presents a similar diagram showing the application of a field gradient sequence, soon after or preferably during magnetized cell injection. Such a sequence in the present invention is used to supplement the forces applied onto the magnetically loaded particles by the implant local gradients, by providing a “macro” gradient on the scale of the implant or vessel diameter. This gradient combines additively with the implant gradients to effectively pull the particles away from the vessel lumen and lumen axis and towards the implant surface and vessel walls. To ensure a radially uniform particle distribution, the gradients are varied in time during the cell injection and distribution to the target area. Such a sequence of fields can be applied using specific electromagnets, or, in the case of the Stereotaxis Niobe™ system, by using the navigation magnets. The Niobe™ system magnets can be used for such an application, even should the catheter or medical device navigation be effected by other means, such as a mechanically controlled navigation system; in that case the Niobe™ permanent magnets would be brought in from a semi-stowed position to perform a magnetization and/or gradient field sequence. An MRI imaging magnet system could also be used to apply such a sequence of gradient fields, as such systems rely on gradient field sequences for image generation. In such an embodiment using gradient magnetic fields, ideally the gradients are orthogonal to the local sent or implant long axis; however in practice that condition is a weak requirement, as the length of the stent or implant is typically several times its diameter; accordingly, the solid angle sustained by the implant or stent at most points along its long axis is large, and even gradients at relatively shallow angles to the local axis will pull magnetized cells away from the lumen center and toward the implant surfaces and organ wall(s). In such an embodiment, the size of the magnetic domains is of limited relevance, as the dominant forces are provided by the applied gradient(s) when the magnetized cells are away from the implant surface, and impart an average speed to the magnetized cells sufficient to ensure contact of the cells with the vessel or organ walls, even in the presence of significant blood flow. Accordingly, in such an embodiment, the cardiac cycle phase during magnetized cell injection is of reduced importance.

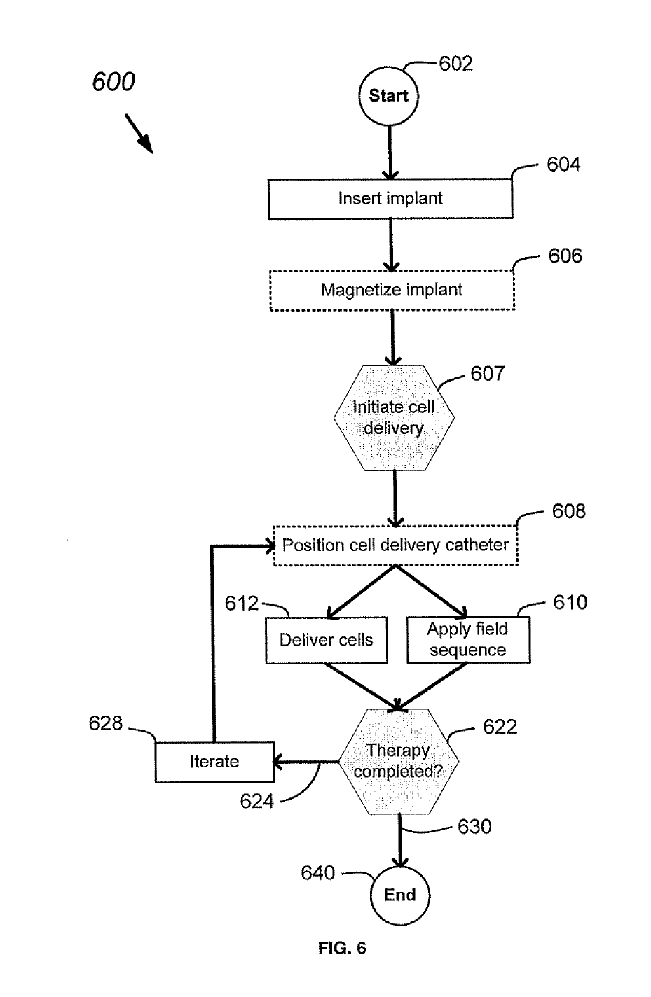

The sequence of steps for magnetic cell delivery according to a preferred embodiment of the present invention is illustrated in FIG. 6 as generally designated by numeral 600. Upon start of the procedure, 602, the pre-magnetized, weakly magnetized, or not-yet magnetized implant or other medical device is delivered 604 to the target area, such as an arterial stenosis. In optional step 606, the implant is magnetized, or further magnetized. Cell delivery 607 is then initiated, the cell delivery catheter distal tip position is adjusted if necessary, step 608, and cells are delivered through the catheter, 612. During delivery, a magnetic field sequence 610 is applied to the implant volume; in a preferred embodiment of the invention, a field gradient sequence is applied to the implant volume, preferably with the fields and their gradients in planes essentially perpendicular to the local implant main axis. The gradients are designed so that the field variations are maximized at the implant or vessel center, to impart maximum applied forces to magnetically particles flowing therethrough; the gradients are designed such that a significant field magnitude remains at the implant surface and/or vessel wall, to impart additional magnetization to the local domains. Following delivery at the local treatment point, the decision point 622 is reached, and if the treatment is completed the method terminates, step 640; otherwise, the method iterates 628 through steps 608, 610, and 612, through completion.

The advantages of the above described embodiments and improvements should be readily apparent to one skilled in the art, as to enabling delivery of cells or similar therapeutic agents or particles to a targeted organ or organ surface. Additional design considerations may be incorporated without departing from the spirit and scope of the invention. Accordingly, it is not intended that the invention be limited by the particular embodiments or forms described above, but by the appended claims.

What is claimed is:

1. A method of using a magnetic system for the delivery of magnetized particles to a target area in a subject, the method comprising:
i) delivering a magnetizable implant to the target area in the subject;
ii) magnetizing the implant and generating local implant magnetic field gradients by applying an externally generated magnetic field to the implant;
iii) inserting a hollow medical device in the subject and navigating the hollow medical device to the vicinity of the magnetized implant;
iv) injecting magnetized particles in the vicinity of the magnetized implant through the hollow medical device; and
v) applying a magnetic field sequence to the implant during the injection of magnetized particles;
whereby the injected magnetized particles are attracted to the implant by the local magnetic field gradients and delivered to the target area in the subject.

2. The method of claim 1, wherein the applied magnetic field sequence comprises fields that are essentially uniform across the target area.

3. The method of claim 2, wherein the applied fields are essentially perpendicular to the axis of the implant.

4. The method of claim 1, wherein the applied magnetic field sequence comprises fields that present a gradient across the target area.

5. The method of claim 4, wherein the applied fields are essentially perpendicular to the axis of the implant.

6. A method of using a magnetic system for the delivery of magnetized particles to a target area in a subject, the method comprising:
i) delivering a magnetized implant to the target area in the subject;
ii) inserting a hollow medical device in the subject and navigating the hollow medical device to the vicinity of the magnetized implant;
iii) injecting magnetized particles in the vicinity of the magnetized implant through the hollow medical device; and
iv) applying a magnetic field sequence to the implant during the injection of magnetized particles;
whereby the injected magnetized particles are attracted to the implant by the local magnetic field gradients and delivered to the target area in the subject.

7. A method of delivering magnetized particles to an organ wall of a subject body, the method comprising:
i) inserting a medical device comprising a hollow lumen in the subject body and guiding the medical device distal tip to the vicinity of the organ wall;
ii) deploying a magnetizable medical implant to contact the organ wall;
iii) magnetizing the medical implant and generating local magnetic field gradients by applying a sequence of magnetic fields to the medical implant;
iv) injecting magnetic particles through the medical device hollow lumen; and
v) applying a magnetic field sequence during the injection;
whereby the magnetized particles are attracted to the implant by the local magnetic field gradients and delivered to the organ wall.

8. The method of claim 7, wherein the applied magnetic field sequence comprises fields that are essentially uniform across the target area.

9. The method of claim 7, wherein the applied magnetic field sequence comprises fields that present a gradient across the target area.

10. A method of delivering magnetized particles to an organ wall of a subject body, the method comprising:
i) delivering a magnetized implant to the target area in the subject;
ii) magnetizing the medical implant and generating local magnetic field gradients by applying a sequence of magnetic fields to the medical implant;
iii) injecting magnetic particles through the medical device hollow lumen; and
iv) applying a magnetic field sequence during the injection;
whereby the magnetized particles are attracted to the implant by the local magnetic field gradients and delivered to the organ wall.

11. A method of delivering therapeutic particles to an target area of a subject body comprising a magnetizable implant, the method comprising:
i) magnetizing the therapeutic particles;
ii) inserting a medical device comprising a hollow lumen into the subject body;
iii) navigating the medical device distal tip to the neighborhood of the magnetizable implant;
iv) injecting the magnetized therapeutic particles at the medical device proximal end; and
v) applying a sequence of magnetic fields to the magnetizable implant during at least part of the therapeutic particles injection.

12. The method of claim 11, wherein the applied magnetic field sequence comprises fields that are essentially uniform across the target area.

13. The method of claim 11, wherein the applied magnetic field sequence comprises fields that present a gradient across the target area.

14. The method of claim 11 further comprising the step of magnetizing the implant prior to the therapeutic particles injection.

15. The method of claim 11 further comprising the step of magnetizing the implant during the therapeutic particles injection.

16. A method of coating a medical implant with a magnetizable alloy, the method comprising:
i) selecting an alloy from the group consisting of platinum cobalt, nickel, platinum-iron, and iron oxides to achieve favorable magnetization response in an applied magnetic field in the range of 0.05 tesla to 5.0 tesla;
ii) selecting an alloy deposition pattern favorable to the generation of local magnetic field gradients; and
iii) depositing a layer of alloy comprising a high-density of local magnetic domains through a method selected from the group comprising electroplating, etching, dip coating, and sputtering.

17. The method of claim 12, further comprising the step of encapsulating the alloy by depositing a bio-compatible polymer on the implant surface.

18. A medical implant for use with a navigation system comprising a magnet, the implant comprising:
i) a grid of sites where a magnetizable alloy is deposited;
ii) a layer of magnetizable alloy at the sites of grid i), the alloy being selected from the group consisting of platinum cobalt, nickel, platinum-iron, and iron oxides;
iii) a layer of bio-compatible material covering non-biocompatible alloy surfaces.
